(19) United States
(12) Patent Application Publication
(10) Pub. No.: US 2025/0246281 A1
(43) Pub. Date: Jul. 31, 2025

(54) MULTI-MODAL, MULTI-OMIC ENTERPRISE GRAPH-BASED, SEMANTIC ONTOLOGY-BASED RECOMMENDER FRAMEWORK

(71) Applicant: CITADEL INFORMATION SERVICES PRIVATE LIMITED, Hyderabad, Telangana (IN)

(72) Inventors: Ramesh MUNNURU, Hyderabad (IN); Srinivas MUNURI, Hyderabad (IN); Reena Ramesh GOLLAPUDY, Hyderabad (IN)

(73) Assignee: CITADEL INFORMATION SERVICES PRIVATE LIMITED, Hyderabad, Telangana (IN)

(21) Appl. No.: 18/853,989

(22) PCT Filed: Apr. 4, 2023
(86) PCT No.: PCT/IB2023/000146

§ 371 Date: Oct. 3, 2024

Related U.S. Application Data
(60) Provisional application No. 63/326,953, filed on Apr. 4, 2022.

(51) Int. Cl. G16H 20/00; G06F 16/901; G16H 10/60; G16H 50/70

(52) U.S. Cl. CPC G16H 20/00; G06F 16/9024; G16H 10/60; G16H 50/70

(57) ABSTRACT

Embodiments herein a multi-modal, multi-omic enterprise, graphformat, semantic ontology-based metadata framework spanning the value continuum across a plurality of technological fields, such as, but not limited to, life sciences, pharma, healthcare, and so on. In an embodiment herein, the framework can be a Cloud implementation. In ah embodiment herein, the framework can be a local implementation. In an embodiment herein, the framework can be a mix of a Cloud implementation and a local implementation.

### CROSS REFERENCE TO RELATED APPLICATION

This application is based on and derives the benefit of U.S. Provisional Patent Application 63/326,953, the contents of which are incorporated herein by reference.

### TECHNICAL FIELD

The embodiments herein relate to information management systems, and, more particularly, to a multi-modal, multi-omic enterprise graph-based, semantic ontology-based recommender framework, wherein the framework can be used in a plurality of technological fields (such as life sciences, pharma, healthcare, and so on).

### BACKGROUND

Fields, such as life sciences, pharma, healthcare, involve large amounts of data. In some cases, this data can be unstructured, as each organization/department/personnel (such as physicians, nurses, pharmacists, and so on) can have their data organization structure to be unique and hence, may not be compatible with each other. Further, performing operations (such as analyzing the data, querying the data, and so on) on the data can be difficult due to the varying organization structure and the large amounts of involved data.

### BRIEF DESCRIPTION OF THE FIGURES

The embodiments disclosed herein will be better understood from the following detailed description with reference to the drawings, in which:

FIG. 1 depicts a system for enabling an enterprise graph-based, semantic ontology-based recommender framework, according to embodiments as disclosed herein;

FIG. 2 depicts the domain architecture, according to embodiments as disclosed herein;

FIG. 3 depicts the architecture of the radiomic pipeline, according to embodiments as disclosed herein;

FIGS. 4A and 4B depict the process of analyzing radiomics data, according to embodiments as disclosed herein;

FIG. 5 depicts the process of training and validating the AI models, according to embodiments as disclosed herein;

FIG. 6 depicts the process of the functioning of the inference engine, according to embodiments as disclosed herein;

FIG. 7 depicts the framework of the pathomic pipeline, according to embodiments as disclosed herein;

FIG. 8 depicts the AI based signal analytical architecture, according to embodiments as disclosed herein;

FIG. 9 depicts the OCR framework, according to embodiments as disclosed herein;

FIG. 10 depicts the process of performing OCR using AI/NLP, according to embodiments as disclosed herein;

FIG. 11 depicts a process of performing AI/NLP based vocabulary categorization from electronic medical record/electronic health record (EMR/EHR), according to embodiments as disclosed herein;

FIG. 12 depicts an example AI based ontology framework for predicting the survival of a patient suffering from cancer, according to embodiments as disclosed herein;

FIG. 13 depicts an example scenario, wherein a 3D image is analyzed, according to embodiments as disclosed herein;

FIG. 14 depicts a system for providing recommendations to a user, in response to a user request, according to embodiments as disclosed herein;

FIGS. 15A and 15B depict a process for providing precision medicine treatment to a patient by comparing to a cohort (i.e., a group of patients), according to embodiments as disclosed herein;

FIGS. 16A and 16B depicts an example ontology for breast cancer and one or more therapies/treatment plans for a patient respectively, wherein the semantic ontology is stored in a graph format, according to embodiments as disclosed herein;

FIG. 17 depicts example radiomic features and predictions of the survival of the patient based on an analysis performed about the survival of the patient, according to embodiments as disclosed herein;

FIG. 18 depicts an example independent unified workspace, according to embodiments as disclosed herein;

FIGS. 19A and 19B depict example screenshots, wherein the linked patent data and the clinical journey of the patient can be viewed by an authorized user, according to embodiments as disclosed herein;

FIGS. 20A, 20B and 20C depict example screenshots, wherein the screenshots enable an authorized user to see data related to a cohort (i.e., a group of patients), view, manage and analyze the data related to the cohort, and the data can be in the form of text and/or graphs, according to embodiments as disclosed herein;

FIG. 21 depicts an example recommendation provided for a patient, according to embodiments as disclosed herein; and

FIGS. 22A, 22B, 22C, 22D, 22E and 22F depict an example overview of the system for providing a recommendation of a prescription to a doctor for a patient, according to embodiments as disclosed herein.

### DETAILED DESCRIPTION OF EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

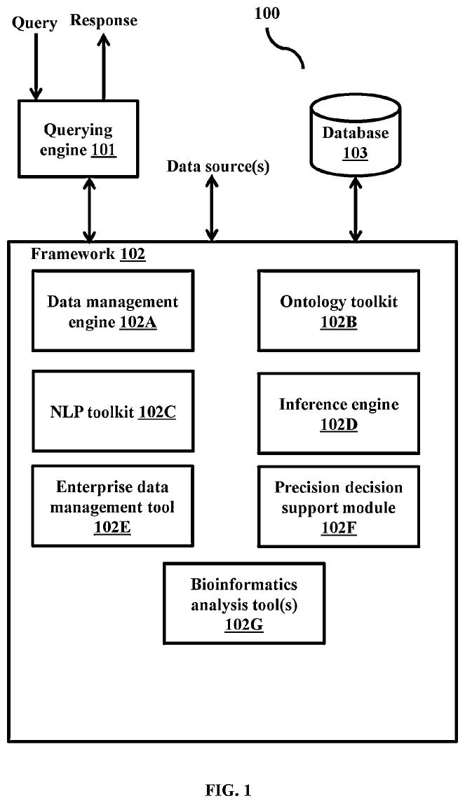

The embodiments herein disclose a multi-modal, multi-omic enterprise graph-based, semantic ontology-based recommender framework, wherein the framework can be used in a plurality of technological fields (such as life sciences, pharma, healthcare, and so on). Referring now to the drawings, and more particularly to FIGS. 1 through 22F, where similar reference characters denote corresponding features consistently throughout the figures, there are shown embodiments.

Embodiments herein a multi-modal, multi-omic enterprise, graph-format, semantic ontology-based metadata framework spanning the value continuum across a plurality of technological fields, such as, but not limited to, life sciences, pharma, healthcare, and so on. In an embodiment herein, the framework can be a Cloud implementation. In an embodiment herein, the framework can be a local implementation. In an embodiment herein, the framework can be a mix of a Cloud implementation and a local implementation.

Multimodal data refers to data that spans different types and contexts (e.g., imaging, text, or genetics). A multimodal causality is important in the precision medicine domain because different modalities can contribute to a result.

Multi-omics data broadly covers the data generated from genomes, proteomes, transcriptomes, metabolomes, and epigenomes. The spectrum of omics can be further extended to other biological data such as lipidome. Multi-omics data generated for the same set of samples can provide useful insights into the flow of biological information at multiple levels and thus can help in unravelling the mechanisms underlying the biological condition of interest.

FIG. 1 depicts a system 100 comprising of the framework 102, a querying engine 101, and at least one database (or a data storage means) 103. The system 100 can be connected to one or more data sources 103. The data present in the data source(s) 103 can be static or real-time data. In an embodiment herein, the framework 102 can be deployed at the data source itself. The data source(s) 103 can comprise of real time data or historical data. The database 103 can be any data storage means, such as, a data server, a file server, the Cloud, and so on.

The framework 102 is an enterprise, graph-format, semantic ontology-based metadata framework and provides a knowledgebase in the form of the enterprise semantic framework. The framework 102 can comprise of modules such as, a data management engine 102A, an ontology toolkit 102B, a Natural Language Processing (NLP) toolkit 102C, an inference engine 102D, an enterprise data management tool 102E, a precision decision support module 102F, and one or more bioinformatics analysis tool(s) 102G. The framework 102 can be modular and comprise of lesser or greater number of modules, than those listed above. The framework 102 has data spaces or data environments available for access to petabytes of data from across data types.

The querying engine 101 can be an Artificial Intelligence/Machine Learning (AI/ML) based dynamic query-response system which leverages the framework 102. The querying engine 101 can accept one or more queries from a user/system (which has been duly authorized) through a user interface (UI), query the framework 102 and provide a response to the user/system through the UI or any other suitable means (wherein the response comprises of data provided by the framework 102 in response to receiving the query from the querying engine 101). In an embodiment herein, the querying engine 101 can be present external to the framework 102. In an embodiment herein, the querying engine 101 can be present internally to the framework 102.

The data management engine 102A can perform various functions such as, automated profiling of the data from the one or more data source(s), performing Quality Assurance/Quality Check (QA/QC) checks on the data, transform and convert data to other formats (such as a graphical format (such as Resource Description Format (RDF)), and so on). The data management engine 102A can also perform Artificial Intelligence/Natural Language (AI/NL) based self-arranging data procedures.

In an embodiment herein, the data management engine 102A can be scalable and can be configured to apply various methods, such as, but not limited to, retrospective analytical algorithms, predictive algorithms, and so on.

In an embodiment herein, the data management engine 102A can automatically augment the data, if there is inadequate data for testing and validation of algorithms/inferences.

The ontology toolkit 102B can be a toolkit for building, maintaining, and standardized ontologies, related to the data being currently analyzed. If the data being analyzed is related to precision medicine, the ontology toolkit 102B can download related ontologies, build ontology process based on the downloaded ontologies, link the ontology processes, and map the common classes arising out of the ontology processes.

The NLP toolkit 102C can process the text present in the data into structured data, wherein the data can be unstructured data (such as prescriptions (which can be typed and/or handwritten), medical charts (which can be typed and/or handwritten) and so on).

The inference engine 102D can draw one or more inferences from the data, wherein the data is received from the data management engine 102A. The inference engine 102D can enable a user to navigate through the visualized data and published contents. The inference engine 102D can enable the user to query the visualized data and published contents and receive one or more responses to the query through the dashboard. The inference engine 102D can process the graphical data to create one or more inferences (along with other modules in the framework, such as the ontology toolkit 102B, the NLP toolkit 102C, and so on) using the semantic ontology framework, which can be further stored in the database 103. Examples of the inference can be, but not limited to, predicting patient outcomes, recommending the potential next best action (such as next treatment process for the patient), and so on.

The enterprise data management tool 102E can read/ingest, cleanse, transform and store the data in a graphical format in the database 103. In an example herein, the enterprise data management tool 102E can read/ingest, cleanse, transform and store the data in RDF in the database 103. The enterprise data management tool 102E can process both static and real-time data prior to either incorporation into the graphical format or as a test dataset to be analyzed.

The precision decision support module 102F can be embedded in the framework 102, and can provide point-of-care deployment to support physicians in their clinical decision-making process.

The one or more bioinformatics analysis tool(s) 1021 can comprise one or more tools, which can be used for understanding the data and extracting knowledge from the data.

The framework 100 can comprise an architecture, which can comprise of one or more dashboards, one or more recommender systems, and the inference engine. The dashboards can be used for visualizing data and publishing one or more contents (such as, but not limited to, graphs) based on the data. The inference engine can enable a user to navigate through the visualized data and published contents. The inference engine can enable the user to query the visualized data and published contents and receive one or more responses to the query through the dashboard.

The framework can comprise one or more data environments. Examples of the data environments can be custom data, consumer data (HCP/patient/HCO), payer data, device data, clinical data, scientific data, and so on.

The framework can comprise a technology services layer. This layer can enable one or more microservices (such as developing algorithms/methods/approaches, testing the developed algorithms, and validating and provisioning the developed algorithms/methods/approaches). The algorithms/methods/approaches can be developed based on one or more inputs received from an authorized user. The technology services layer can comprise of a query processing engine. The query processing engine can receive one or more queries, and provide a response to the received queries. For example, the query processing engine can use Kafka messaging for managing the queries.

There can be one or more domains and use cases for providing one or more domain services. This can comprise of use case definitions, and a validation engine. This can enable testing, benchmarking, and optimization of the developed algorithms/methods/approaches across the various domains.

The framework can comprise a datastore, which can be an enterprise integration engine (ETL/NLP/RDF conversions) (which can acquire and aggregate data), a data store (which can store reference/raw data), and a data processing engine (which can involve the ontology metadata framework).

In an embodiment herein, the framework 100 can perform load balancing automatically. In an embodiment herein, the framework 100 can perform auto-scaling resources required for operations of the framework automatically.

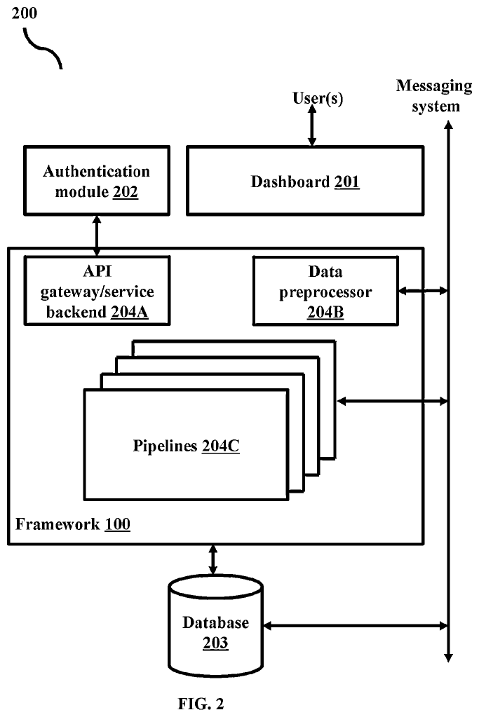

FIG. 2 depicts the domain architecture. The architecture 200 comprises a dashboard 201, the framework 100, an authentication module 202, and one or more databases 203. The architecture 200 can comprise a messaging system, which enables the components of the framework to communicate with each other and the other components of the architecture.

The dashboard 201 can comprise one or more UIs and/or interfaces. The dashboard 201 can enable one or more users to interact with the framework 100 using a graphical UI, and/or a command line directly or through one or more user devices.

The framework 100 can comprise an Application Programming Interface (API) gateway/service backend 204A, a data preprocessor 204B, and one or more pipelines 204C (such as a clinical pipeline, a radiomic pipeline, a pathomic pipeline, and a genomic pipeline). The clinical pipeline can manage data arising out of medical tests, laboratory results, laboratory tests, medical reports, and so on. The radiomic pipeline can manage data arising out of media (such as images, videos, animations, and so on) from medical scans. The pathomic pipeline can manage media (such as images, videos, animations, and so on) from the study of cells and tissues.

The authentication module 202 can provide functions, such as identity and access management. The authentication module 202 can authenticate users, before providing access to the user, only after successfully authenticating the user.

The one or more databases 204 can comprise data, wherein the data can comprise of raw data (i.e., medical data, as provided by the respective stakeholders), processed data, converted data, graphical data, metadata, and so on. The data can be stored in a suitable data structure. In an embodiment herein, the processed data can be stored as graphical data. In an embodiment herein, the database 204 can be a relational database.

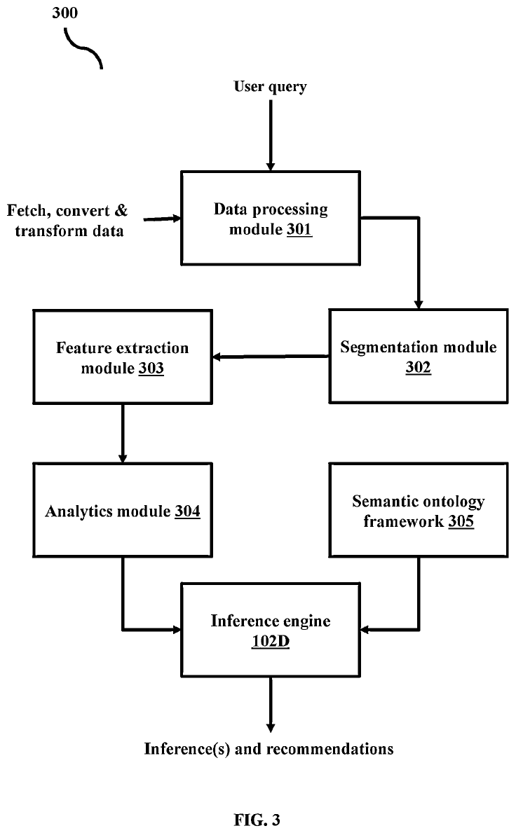

FIG. 3 depicts the architecture of the radiomic pipeline. The radiomic pipeline can manage data arising out of media (such as images, videos, animations, and so on) from medical scans to provide recommendations related to precision medicine. The architecture 300 can collect raw, unstructured data, which can be filtered further. In an embodiment herein, the data can be in the form of radiological media. The data can be converted to a suitable uniform format, such as, but not limited to, NIfTI (Neuroimaging Informatics Technology Initiative). Additionally, metadata can also be extracted from the media. The media can be checked for quality, such as, the contrast levels (i.e., the media should have sufficient contrast levels, so as to identify the organs, tissues, bones, muscle etc.), presence/absence of the entire organ, presence/absence of artifacts, and so on. The filtered data can then be converted to structured data (using NLP and/or Optical Character Recognition (OCR)). In an embodiment herein, the converted data can be anonymized. The data can then be provided to the radiomic framework using the messaging system.

The pipeline can comprise of a data processing module 301, a segmentation module 302, a feature extraction module 303, and an analytics module 304. The data processing module 301 can identify comorbidity expressions in the media. The data processing module 301 can further identify coexistence of abnormalities in the media. The data processing module 301 can further identify one or more Region of Interests (ROIs) in the media. Examples of the ROIs can be, but not limited to, one or more organs of interest, one or more anatomical structures of interest, semantic features of interest, and so on. The data processing module 301 can identify relevant radiological/pathological parameters in the data; such as, but not limited to, contrast type, stain type, and so on.

The segmentation module 302 can determine one or more segments in the curated data, based on one or more relevant radiological parameters. In an embodiment herein, the segmentation module 302 can determine one or more segments in the curated media, based on one or more relevant radiological parameters manually. In an embodiment herein, the segmentation module 302 can determine one or more segments in the curated media, based on one or more relevant radiological parameters using an AI model, wherein the AI model has been trained and validated.

The feature extraction module 303 can extract one or more features from the segmented data. The feature extraction module 303 can convert the voxel masks (i.e., points in the segmented data) into triangulated and polygonal meshes. The feature extraction module 303 can extract one or more geometrical parameters of semantic features. In an embodiment herein, the feature extraction module 303 can extract one or more geometrical parameters of semantic features using pymeshlab. In an embodiment herein, the feature extraction module 303 can extract one or more geometrical parameters of semantic features using vtk. In an embodiment herein, the feature extraction module 303 can extract one or more geometrical parameters of semantic features using VMTK. In an embodiment herein, the feature extraction module 303 can extract one or more geometrical parameters of semantic features using ITK. The feature extraction module 303 can further verify the robustness of the extracted features. In an example herein, the feature extraction module 303 can verify the robustness of the extracted features using features extracted from an alternate methodology, such as, but not limited to, PyRadiomics.

In an embodiment herein, the feature extraction module 303 can verify the robustness of the extracted features using 3D printing. This process involves using biomaterials (that can mimic tissue, bone, muscles, plasma, blood, tumours, and so on) for 3D printing the organ/part of the body that is being analyzed. The 3D printing can include varied imaging protocols (covariates) with fixed parameters. The 3D printing can further include one or more artefacts. The feature extraction module 303 can determine the robustness using statistical analysis of stability, reproducibility, and generalization of the features by comparing the extracted features and the data from the 3D printed model.

The analytics module 304 can predict outcomes, such as predicting clinical outcomes from the extracted features. In an embodiment herein, the analytics module 304 can predict the clinical outcomes from the extracted features manually. In an embodiment herein, the analytics module 304 can predict the clinical outcomes from the extracted features using computer vision. The predicted clinical outcomes can be provided to the inference engine 102D.

The inference engine 102D can draw one or more inferences from the extracted features using the semantic ontology framework 305. The inference engine 102D can enable a user to navigate through the visualized data and published contents. The inference engine 102D can enable the user to query the visualized data and published contents and receive one or more responses to the query through the dashboard. The inference engine 102D can process the graphical data to generate one or more inferences and recommendation using a semantic ontology metadata framework (along with other modules in the framework, such as the ontology toolkit 102B, the NLP toolkit 102C, and so on), which can be further stored in the database 103.

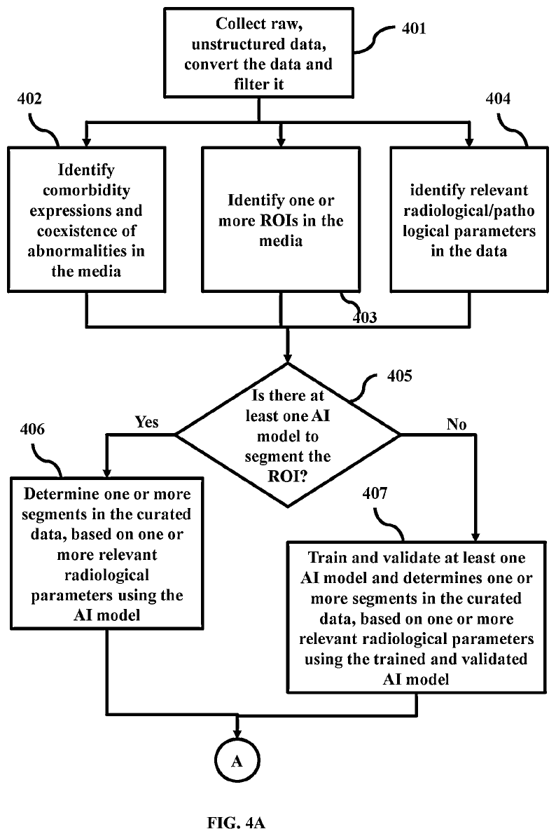

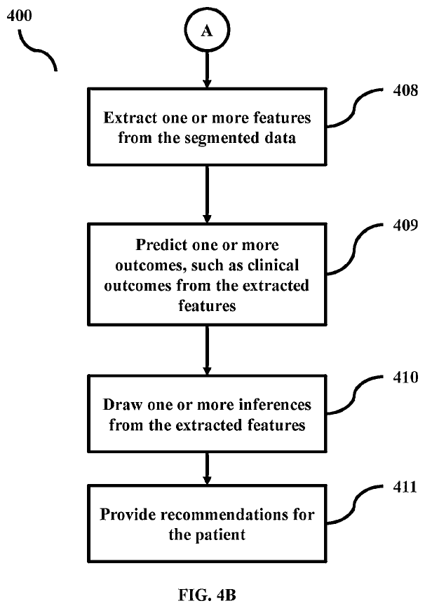

FIGS. 4A and 4B depict the process of analyzing radiomics data. In step 401, the architecture 300 collects raw, unstructured data, which can be converted to a suitable uniform format and filtered. In an embodiment herein, the data can be in the form of radiological media. Additionally, metadata can also be extracted from the media. The media can be checked for quality, such as, the contrast levels, presence/absence of the entire organ, presence/absence of artifacts, and so on. The filtered data can then be converted to structured data (using NLP and/or Optical Character Recognition (OCR)). The converted data may further be anonymized. The data can then be provided to the radiomic framework using the messaging system.

In step 402, the data processing module 301 identifies comorbidity expressions and coexistence of abnormalities in the media. In step 403, the data processing module 301 further identifies one or more ROIs in the media. In step 404, the data processing module 301 identifies relevant radiological/pathological parameters in the data; such as, but not limited to, contrast type, stain type, and so on.

In step 405, the segmentation module 302 checks if there is at least one AI model to segment the ROI. If there is at least one AI model to segment the ROI, in step 406, the segmentation module 302 determines one or more segments in the curated data, based on one or more relevant radiological parameters using the AI model. If at least one AI model to segment the ROI is not available, in step 407, the segmentation module 302 trains and validates at least one AI model and determines one or more segments in the curated data, based on one or more relevant radiological parameters using the trained and validated AI model. Further, the segmentation module 302 may enable at least one authorized user (such as a radiologist) to perform the segmentation manually.

In step 408, the feature extraction module 303 extracts one or more features from the segmented data. In step 409, the analytics module 304 predicts one or more outcomes, such as clinical outcomes from the extracted features, which can be provided to the inference engine 102D. In step 410, the inference engine 102D draws one or more inferences from the extracted features and the semantic ontology framework 305 and provides recommendations for the patient (step 411). The various actions in method 400 may be performed in the order presented, in a different order or simultaneously. Further, in some embodiments, some actions listed in FIGS. 4A and 4B may be omitted.

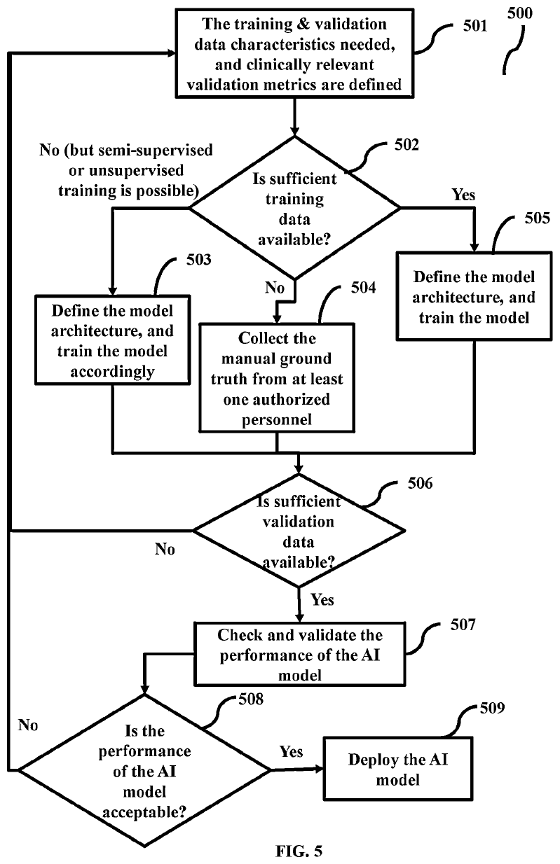

FIG. 5 depicts the process of training and validating the AI models. In step 501, the training & validation data characteristics needed, and clinically relevant validation metrics are defined. In step 502, it is checked if sufficient training data is available. If sufficient training data is not available (but semi-supervised or unsupervised training is possible), in step 503, the model architecture is defined, and the model is trained accordingly. If sufficient training data is not available, in step 504, the manual ground truth is collected from at least one authorized personnel (such as, a radiologist). If sufficient training data is available, in step 505, the model architecture is defined, and the model is trained. In step 506, it is checked if sufficient validation data is available. If sufficient validation data is available, in step 507, performance of the AI model is validated and checked. In step 508, a check is made if the performance of the AI model is acceptable by comparing the performance with one or more pre-defined quality parameters. If the performance of the AI model is not acceptable, the process from step 501 is repeated. If the performance of the AI model is acceptable, in step 509, the AI model is deployed. The various actions in method 500 may be performed in the order presented, in a different order or simultaneously. Further, in some embodiments, some actions listed in FIG. 5 may be omitted.

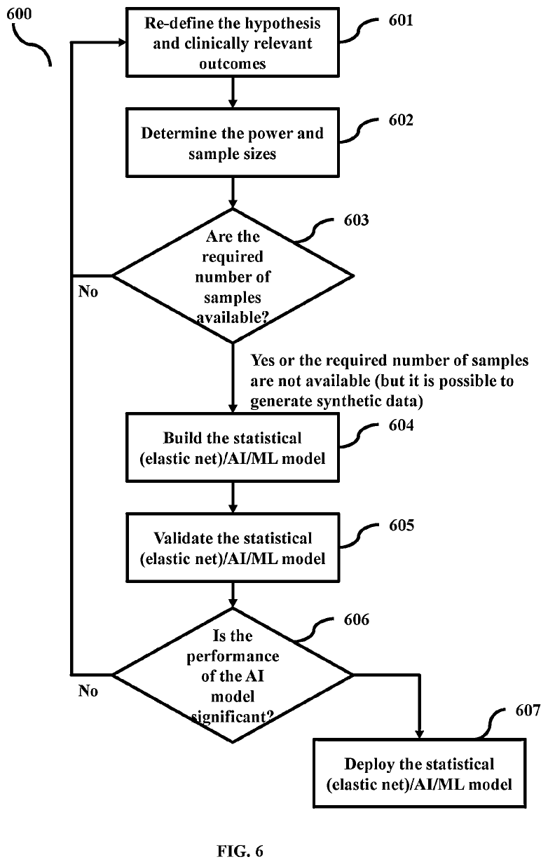

FIG. 6 depicts the process of the functioning of the inference engine. FIG. 6 depicts the iterative nature of AI model applicability and suitability for the user's question/hypothesis under consideration. In step 601, the hypothesis and clinically relevant outcomes are re-defined. In step 602, the power and sample sizes are determined. In step 603, a check is made if the required number of samples are available. If the required number of samples are not available, the process from step 601 is repeated. If the required number of samples are available or the required number of samples are not available (but it is possible to generate synthetic data), in step 604, the statistical (elastic net)/AI/ML model is built. In step 605, the model is validated. In step 606, a check is made if the performance of the AI model is significant by comparing the performance with one or more pre-defined quality parameters. If the performance of the AI model is not significant, the process from step 601 is repeated. If the performance of the AI model is significant, in step 607, the AI model is deployed. The various actions in method 600 may be performed in the order presented, in a different order or simultaneously. Further, in some embodiments, some actions listed in FIG. 6 may be omitted.

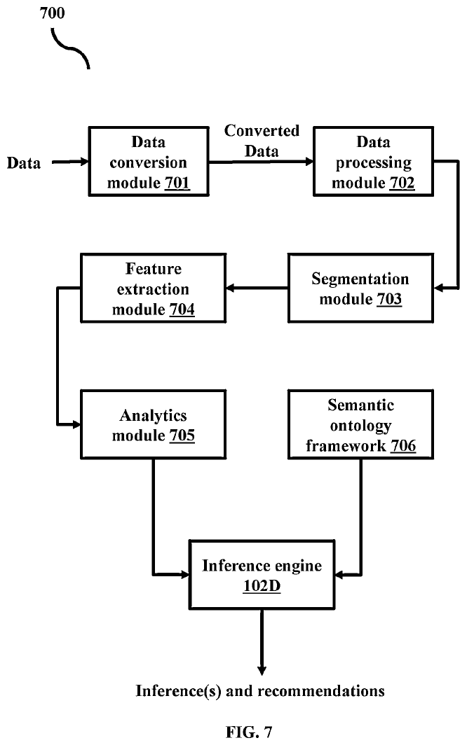

FIG. 7 depicts the framework of the pathomic pipeline. The pathomic pipeline can manage media (such as images, videos, animations, and so on) from the study of cells and tissues. The architecture 700 can collect raw, unstructured data and provided to a data conversion module 701. In an embodiment herein, the data can be in the form of radiological media. The data conversion module 701 can then convert the data to a suitable uniform format, such as, but not limited to, numpy, which can be provided to a data processing module 702.

The data processing module 702 can use the converted media to identify one or more relevant pathological parameters. The data processing module 702 can further identify one or more ROIs in the media. Examples of the ROIs can be, but not limited to, one or more organs of interest, one or more anatomical structures of interest, semantic features of interest, and so on. The data processing module 702 can identify comorbidity expressions in the media. The data processing module 702 can further identify coexistence of abnormalities in the media. The curated data can be provided to a segmentation module 703.

The segmentation module 703 can determine one or more segments in the curated data, based on one or more relevant pathological parameters. In an embodiment herein, the segmentation module 703 can determine one or more segments in the curated media, based on one or more relevant pathological parameters manually. In an embodiment herein, the segmentation module 703 can determine one or more segments in the curated media, based on one or more relevant pathological parameters using the AI model, wherein the AI model has been trained and validated. In an embodiment herein, the segmentation module 703 can train and validate one or more AI models, wherein the segmentation module 703 can determine one or more segments in the curated media, based on one or more relevant pathological parameters using the trained and validated AI model. The segmentations are provided to the feature extraction module 704.

The feature extraction module 704 can extract one or more features from the segmented data. The feature extraction module 704 can extract one or more features from the segmented data from volumetric pixels and triangulated and polygonal meshes.

The analytics module 705 can predict one or more pathological outcomes, such as, but not limited to, Tumor-Infiltrating Lymphocytes (TIL) scores. In an embodiment herein, the analytics module 705 can predict one or more pathological outcomes, such as, but not limited to, TIL scores using hand crafted pathomics (i.e., AI/conventional ML). In an embodiment herein, the analytics module 705 can predict one or more pathological outcomes, such as, but not limited to, TIL scores using deep pathomics (i.e., computer vision AI). The predicted clinical outcomes can be provided to the inference engine 102D.

The inference engine 102D can draw one or more inferences from the extracted features and/or the semantic ontology framework 705 and provide one or more inference(s) and recommendations; i.e., provide a clinical (precision) decision support system. The inference engine 102D can enable a user to navigate through the visualized data and published contents. The inference engine 102D can enable the user to query the visualized data and published contents and receive one or more responses to the query through the dashboard. The inference engine 102D can process the graphical data to create a semantic ontology metadata. The functioning of the inference engine 102D is explained in further detail in FIG. 6.

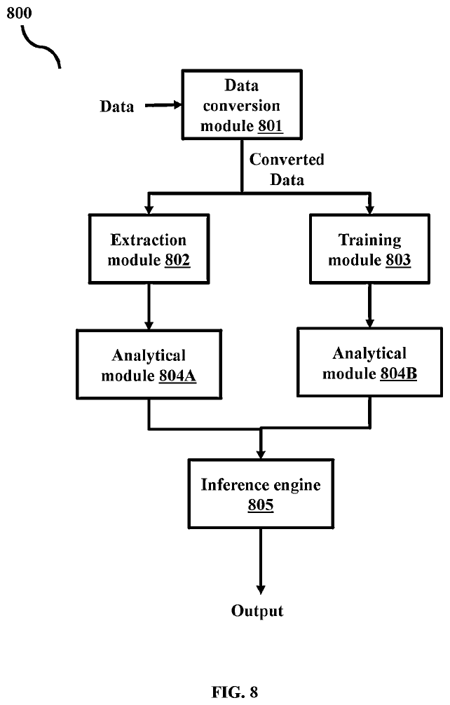

FIG. 8 depicts the AI based signal analytical architecture. The architecture 800 can collect raw, unstructured data and provided to a data conversion module 801. In an embodiment herein, the data can be in the form of media (such as, but not limited to, xml, bmp, jpg, gif, avi, and so on). The data conversion module 801 can then convert the data to a suitable uniform format, such as, but not limited to, numpy, which can be provided to an extraction module 802. The extraction module 802 can extract one or more mathematical features from the converted data, in both the time and frequency domains. The extracted mathematical features are analyzed by an analytical module 804A using one or more handcrafted signal features. Further, the converted data can be provided to a training module 803. The training module 803 can use the converted data to train the Convolutional Neural Network (CNN) and long short-term memory networks/Gated Recurrent Unit (LSTM)/GRU) models. The analytical module 804B can analyze the data using the trained models and one or more deep signal features. The analytics from the analytics module 804A, 804B can be provided to an inference engine 805. The inference engine 805 can draw one or more inferences from the extracted features. The inference engine 805 can enable a user to navigate through the visualized data and published contents. The inference engine 805 can enable the user to query the visualized data and published contents and receive one or more responses to the query through the dashboard. The inference engine 805 can process the graphical data to create semantic ontology metadata.

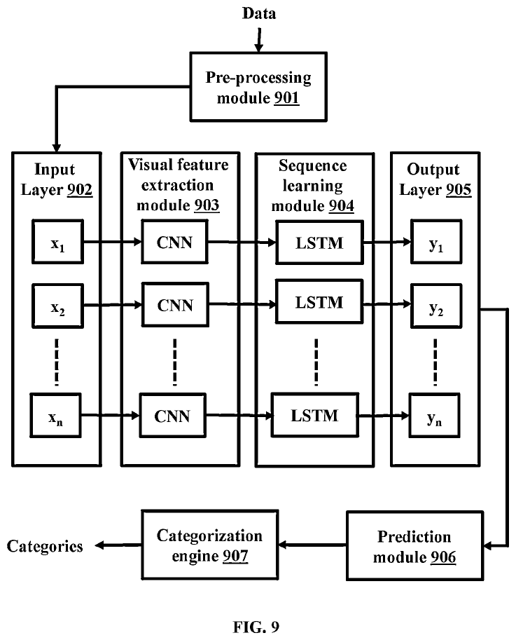

FIG. 9 depicts the OCR framework. The OCR framework 800 can process one or more clinical notes, such as, physician notes, prescription, patient notes/reports, and so on into one or more categories. The clinical notes can be processed by a pre-processing module 901, wherein the pre-processing module 901 can perform operations, such as, but not limited to, scanning the notes, converting the scanned notes into a uniform format, and so on. The processed data can be processed by an input layer 902 (which comprises of n layers), a visual feature extraction module 903 (which can comprise of n CNNs), a sequence learning module 904 (which can comprise of n LSTMs) and an output layer 905 (which comprises of n layers), wherein the output is in the form of a structured output. A prediction module 906 can use the structured output for predicting text present in the notes. In an embodiment herein, the prediction module 906 can use the structured output for predicting the text using a nearest neighbour prediction method. A categorization engine 907 can categorize the predicted text into one or more pre-defined categories. In an embodiment herein, the categorization engine 907 can categorize the predicted text into one or more pre-defined categories using a suitable approach, such as, Named-Entity Recognition (NER).

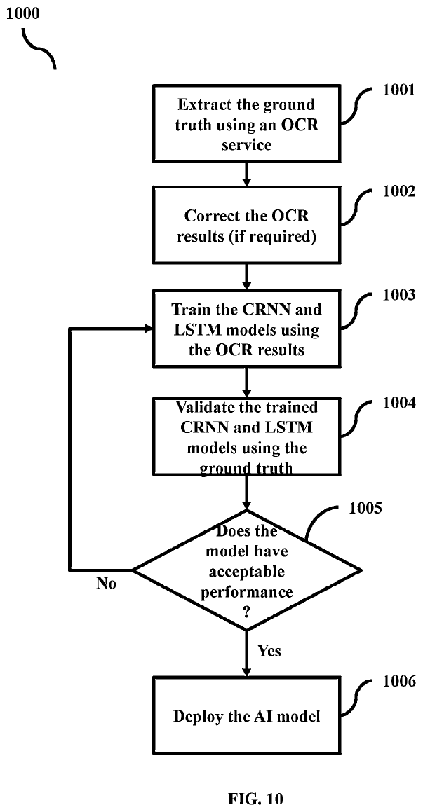

FIG. 10 depicts the process of performing OCR using AI/NLP. In step 1001, the ground truth is extracted using an OCR service, wherein a selected set of handwritten notes is provided as training data. In step 1002, the results of the OCR can be manually corrected (if required). In step 1003, the convolutional recurrent neural network (CRNN) and LSTM models are trained using the OCR results. In step 1004, the trained CRNN and LSTM models are validated using the ground truth. In step 1005, a check is made if the model has acceptable performance. If the model does not have acceptable performance, steps 1003 onwards are repeated, till the model has acceptable performance. If the model has acceptable performance, in step 1007, the model is deployed, wherein the model can be used to categorize the text (as identified in the OCR) into one or more categories. The various actions in method 1000 may be performed in the order presented, in a different order or simultaneously. Further, in some embodiments, some actions listed in FIG. 10 may be omitted.

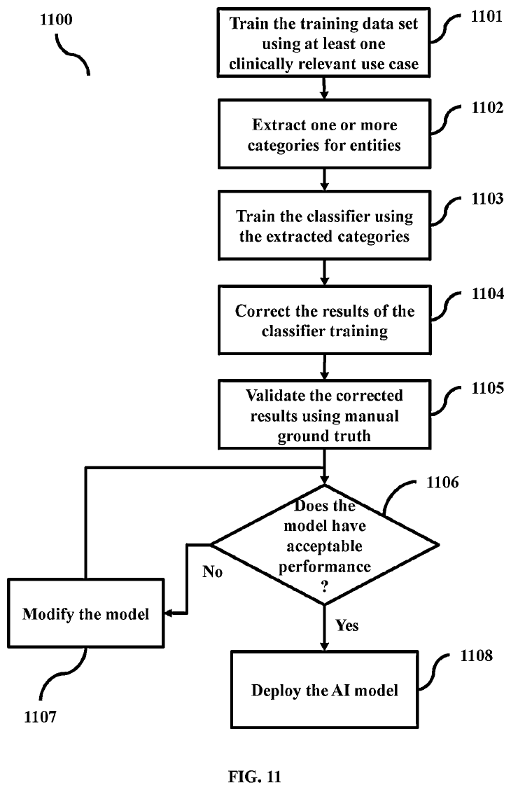

FIG. 11 depicts a process of performing AI/NLP based vocabulary categorization from electronic medical record/electronic health record (EMR/EHR). In step 1101, a training data set is trained using at least one clinically relevant use case, wherein the data set is a selected set of EMR/EHR data. Using the training data set, in step 1102, one or more categories are extracted for entities. In an embodiment herein, the one or more categories can be extracted based on Observational Medical Outcomes Partnership (OMOP). In step 1103, a classifier is trained using the extracted categories. In an example herein, Google BioBert model classifier can be trained. In step 1104, the results of the classifier training can be corrected (if required). In an embodiment herein, the results of the classifier training can be corrected manually. In an embodiment herein, the corrected results can be used as ground truth (GT) for the classifier training. In step 1105, the corrected results are validated using manual ground truth. In step 1106, it is checked if the model has acceptable performance. If the model does not have acceptable performance, in step 1107, the model is modified, the steps 1101 onwards are repeated, till the model has acceptable performance. If the model has acceptable performance, in step 1108, the model is deployed. The various actions in method 1100 may be performed in the order presented, in a different order or simultaneously. Further, in some embodiments, some actions listed in FIG. 11 may be omitted.

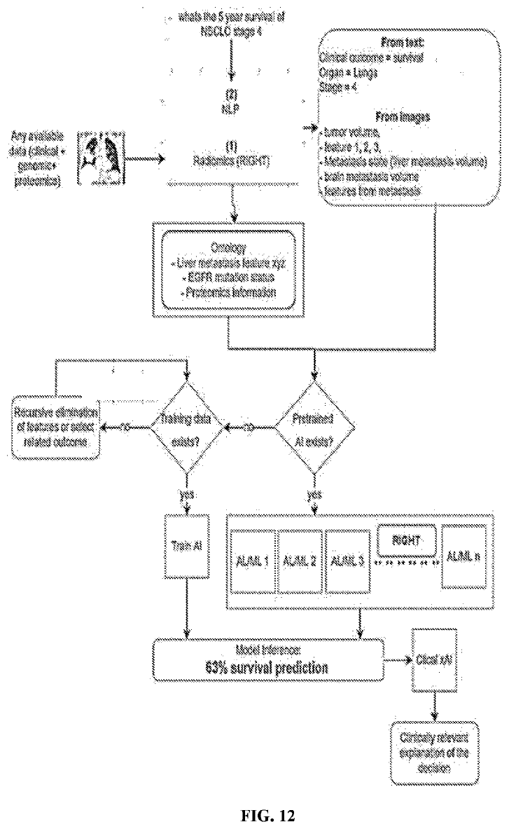

FIG. 12 depicts an example AI based ontology framework for predicting the survival of a patient suffering from cancer. Once the user types in the query, it is converted to a structured query through NLP. Embodiments herein generate one or more insights from radiology, genomics, proteomics and data from other modalities. The clinical and the radiology report generate insights such as clinical outcomes like survival time. The clinical and the radiology report can also generate radiomic features such as tumour volume and metastatic features. Once the features are generated, the underlying radiomic ontology provides knowledge on additional knowledge on additional metastases, estimated glomerular filtration rate (EGFR) mutation status (if applicable) etc. Together, the features and the inputs from the radiology knowledgebase, predictive AI models receive the insights as an input. Embodiments herein can use pretrained AI models or the features can be leveraged to build new AI models. The AI models thus built, in conjunction with the multimodal signatures generated from clinical and other modalities are tested for their ability to explain the end outcomes predicted by the AI model. This is important since AI-based predictions have to be clinically relevant and explainable.

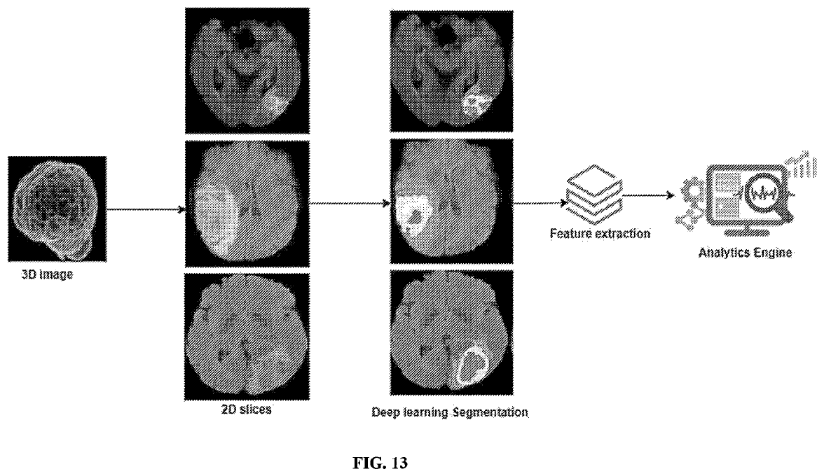

FIG. 13 depicts an example scenario, wherein a 3D image is analyzed. Consider that a 3D image of a patient's brain is provided as input. The patient's brain is sliced into a plurality of two dimensional (2D) slices. Deep learning segmentation is performed on the 2D slices, wherein the segmentation can denote the potential areas where cancer has been detected. The potential areas can be colour coded, wherein the colour can indicate the status/stage of the cancer. One or more features are extracted from the segmented slices, and provided for further analytics, such as, but not limited to, predicting the stage of the cancer, potential therapies/medicines/drugs to be used for treating the patient, determining his survivability rate, and so on.

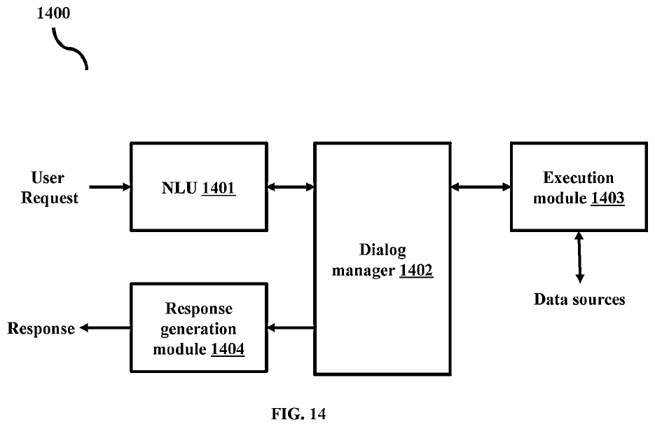

FIG. 14 depicts a system for providing recommendations to a user, in response to a user request. The system 1400, as depicted, comprises a Natural Language Unit (NLU) 1401, which can receive a user request (wherein a user can provide the user request using a UI) from the user. The NLU 1401 can determine the user intent using natural language processing, wherein the NLU 1401 can further use context information related to the user and the query for this. The NLU 1401 can provide the determined user intent to a dialog manager 1402. The dialog manager 1402 can provide a list of instructions to other parts of the system 1400 (such as the execution module 1403), wherein the instructions can be in a semantic representation. The execution module 1403 can execute one or more actions, wherein the actions can depend on the user input using information retrieved from one or more data sources (such as a database, a data storage, a knowledge base, the Internet, an Intranet, and so on). In an example, consider that the user has provided queries related to a patient suffering from cancer; the actions can be, but not limited to, demographic data (which can be in the form of graphs (such as pie chart, bar graph, and so on), categories (such as, but not limited to, tumor location, position, subtypes, deriving cancer staging by individual TNM stages (wherein T describes the size of the tumor and any spread of cancer into nearby tissue; N describes spread of cancer to nearby lymph nodes; and M describes metastasis (spread of cancer to other parts of the body)), tumor grading, etc.), Histologic subtype, molecular subtype, oncotype score (where applicable), surgeries with the type of surgery, prescription/therapy and their order (for example, chemotherapy>radiation>hormone therapy>targeted therapy) (which can be summarized as distinct bars, highlighting how many were neo and how many were adjuvant), predictions of neoadjuvant therapy response, tumor response, recurrence, estimating risk, and so on. The results of these actions can be provided to the user through the dialog manager 1402 and a response generation module 1404. The response generation module 1404 can provide data to the user in one or more formats, which can either be default or in a user defined format. In an embodiment herein, the data can be provided in the form of graphical data, such as pie charts, bar graphs, Venn diagrams, and so on.

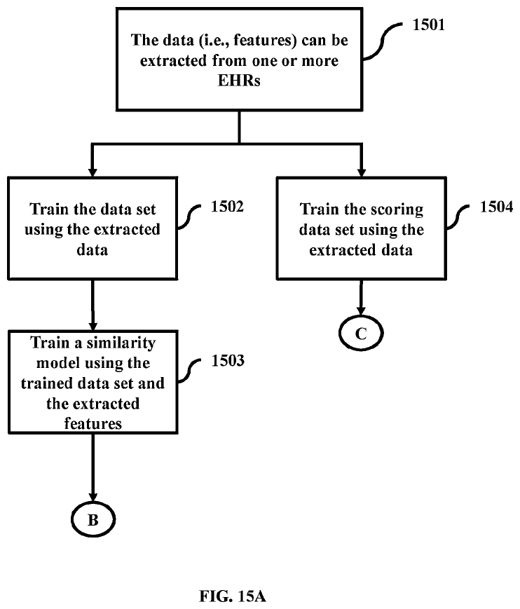

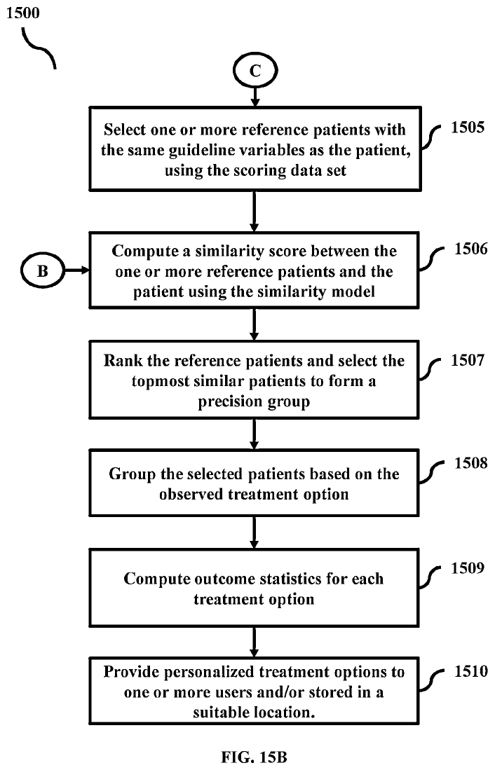

FIGS. 15A and 15B depict a process for providing precision medicine treatment to a patient by comparing to a cohort (i.e., a group of patients). FIG. 15A depicts the training phase, wherein the AI model is trained using a sematic ontology framework. FIG. 15B depicts the runtime phase, wherein the AI model provides recommendations on personalized treatment plans for the patient (i.e., precision medicine).

In step 1501, data (i.e., features) is extracted from one or more EHRs. The extracted features can comprise of identifying clinical decision points, identifying variables from treatment guidelines, identifying variables from the EHRs, and selecting one or more salient variables. Clinical decision points are points in time in the patient longitudinal record where: (a) the disease is not under control, and (b) a treatment decision is warranted. Identifying variables from treatment guidelines associated with the propensity of receiving treatment involves identifying relevant treatment guideline documents, identifying all possible medication treatment options, and identifying one or more patient characteristics used in treatment decisions. Identifying variables from EHRs involves defining at least one appropriate observation window for variables, defining one or more constructor templates for different variable types and implementing and running extraction on EHR data. Selecting salient variables associated with outcomes of interest involves identifying one ore more candidates using a stability ranking feature selection, performing a clinical review and final selection of variables. Step 1501 can be performed at least one of automatically, manually and/or a combination of both.

In step 1502, a data set is trained using the extracted data. In step 1503, using the trained data set and the extracted features, a similarity model is trained, wherein the similarity model can be used to determine the degree of similarity between a patient and a group of patients (who may be having/had similar symptoms, diagnosis, treatment plans, therapy schedule, and so on). In step 1504, a scoring data set is trained using the extracted data, wherein the scoring dataset can enable to determine similarity between the patient and other patients (reference patients).

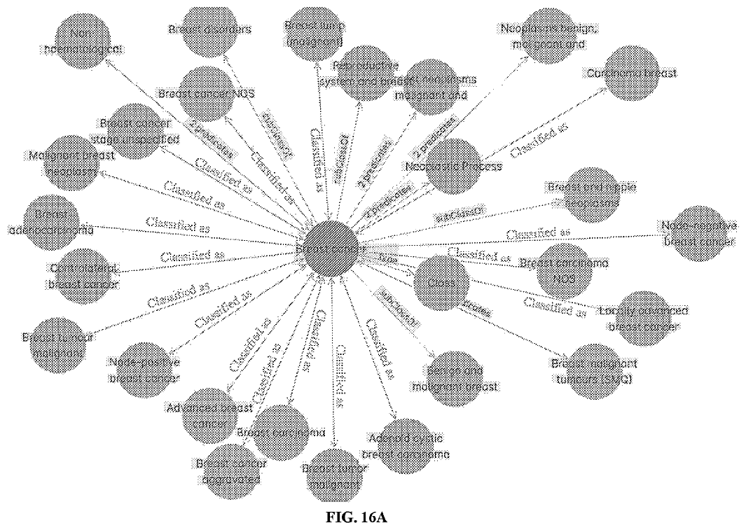

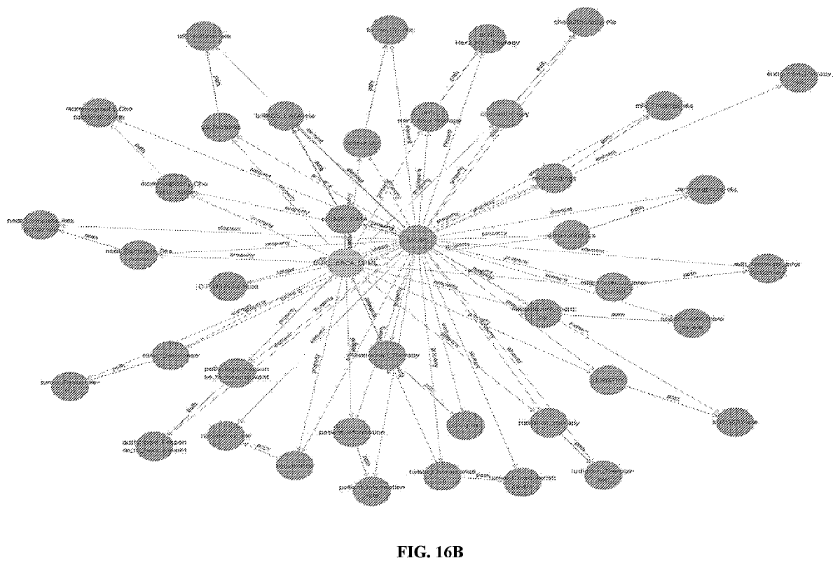

FIGS. 16A and 16B depicts an example ontology for breast cancer and one or more therapies/treatment plans for a patient respectively, wherein the semantic ontology is stored in a graph format. In the depicted example, the relationships between the various terms, such as, but not limited to, multiple classifications, categories, predicates, and so on. FIG. 16B depicts various treatment plans/therapies/drugs that can be provided to the patient, along with additional statistics (such as recurrence, survival rate, and so on).

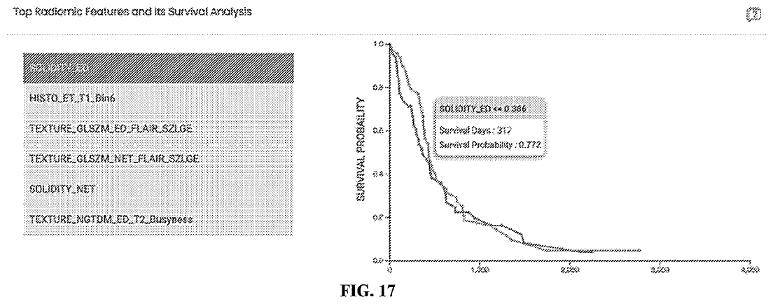

FIG. 17 depicts example radiomic features and predictions of the survival of the patient based on an analysis performed about the survival of the patient.

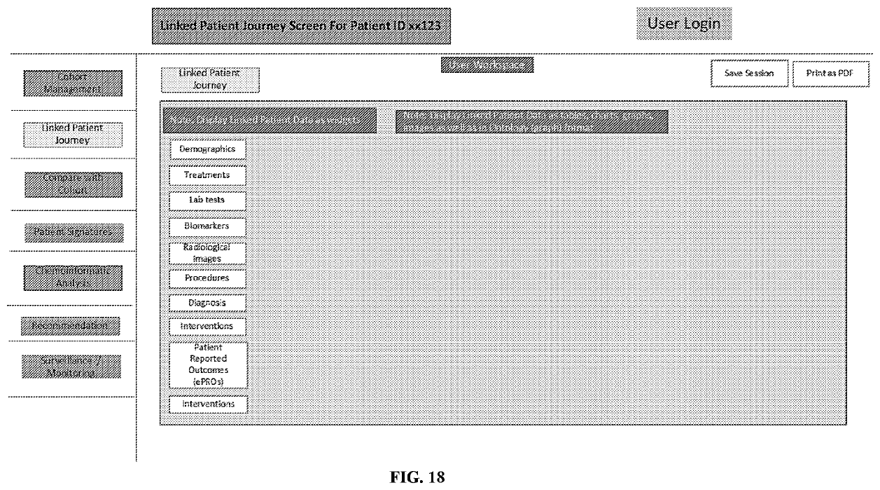

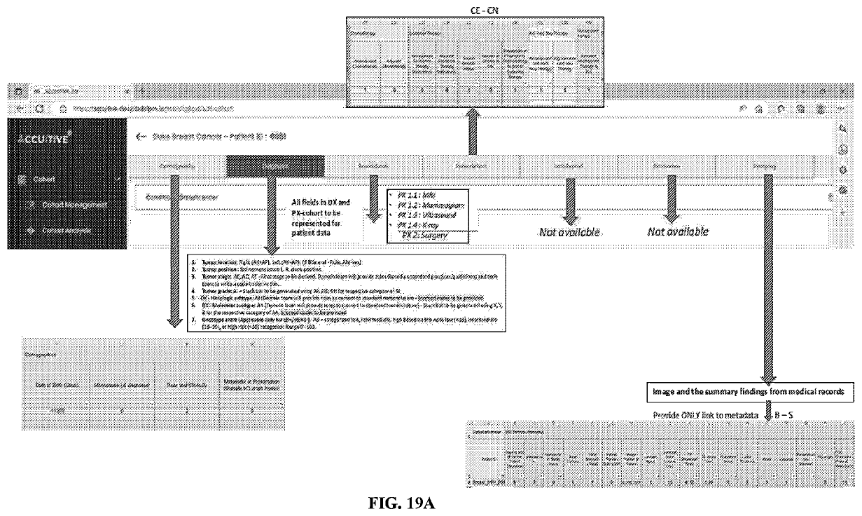

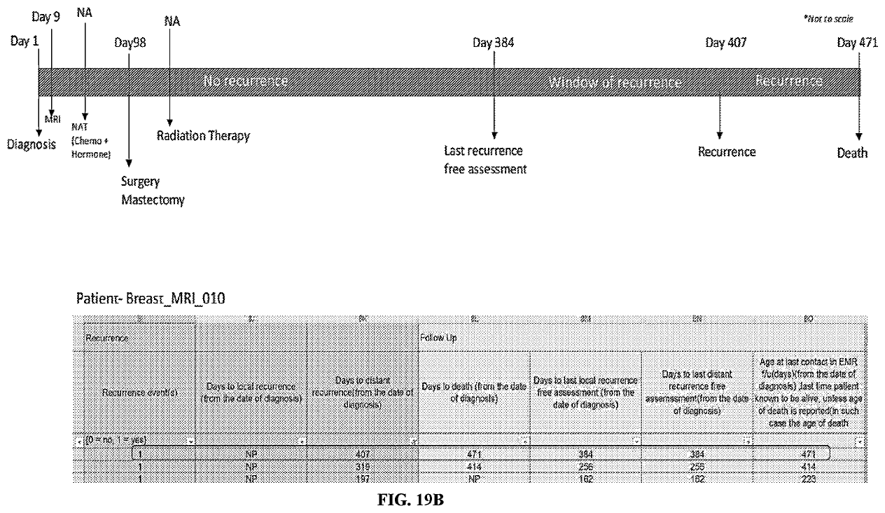

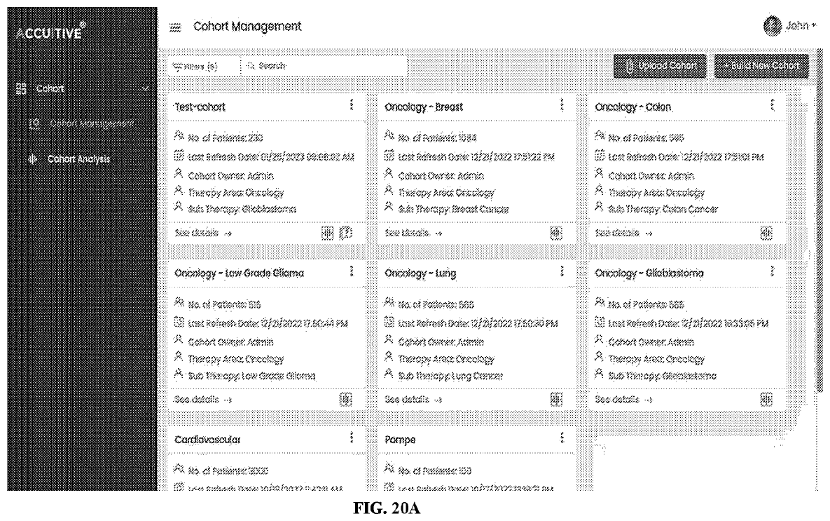

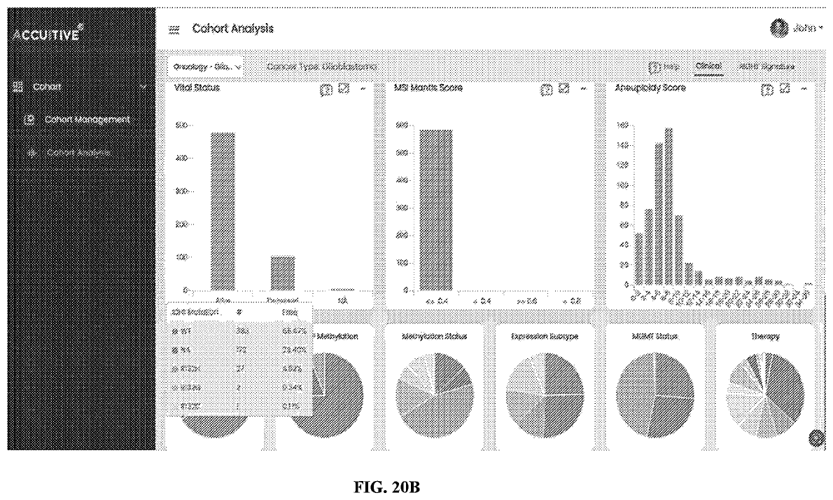

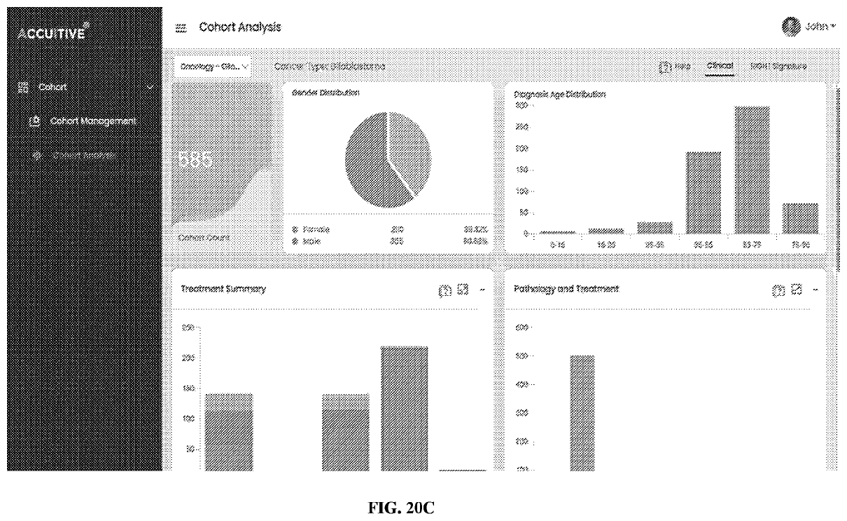

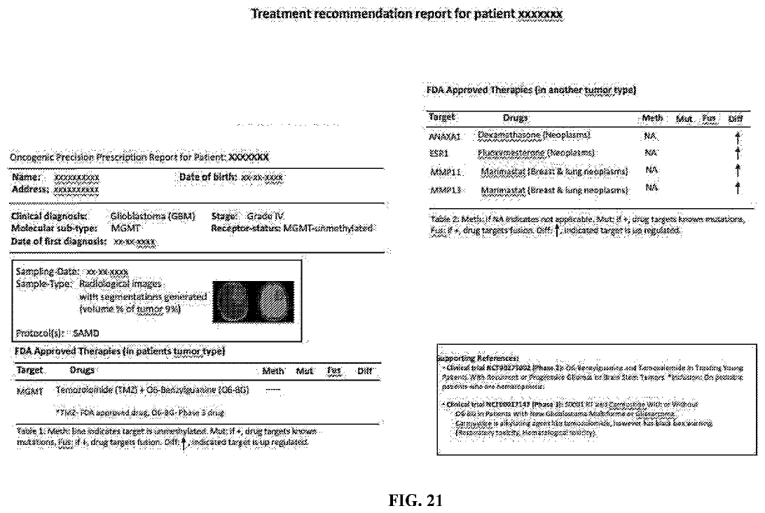

FIG. 18 depicts an example independent unified workspace. The workspace can enable an authorized user to see data related to a single patient in an independent unified workspace. FIGS. 19A and 19B depict example screenshots, wherein the linked patent data and the clinical journey of the patient can be viewed by an authorized user. FIGS. 20A, 20B and 20C depict example screenshots, wherein the screenshots enable an authorized user to see data related to a cohort (i.e., a group of patients), view, manage and analyze the data related to the cohort, and the data can be in the form of text and/or graphs. FIG. 21 depicts an example recommendation provided for a patient.

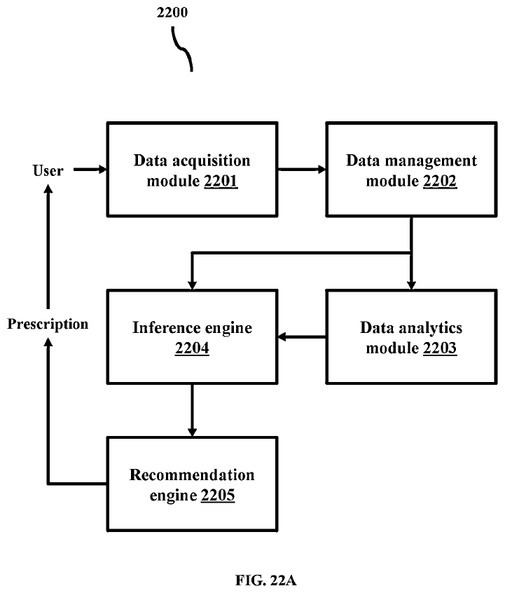

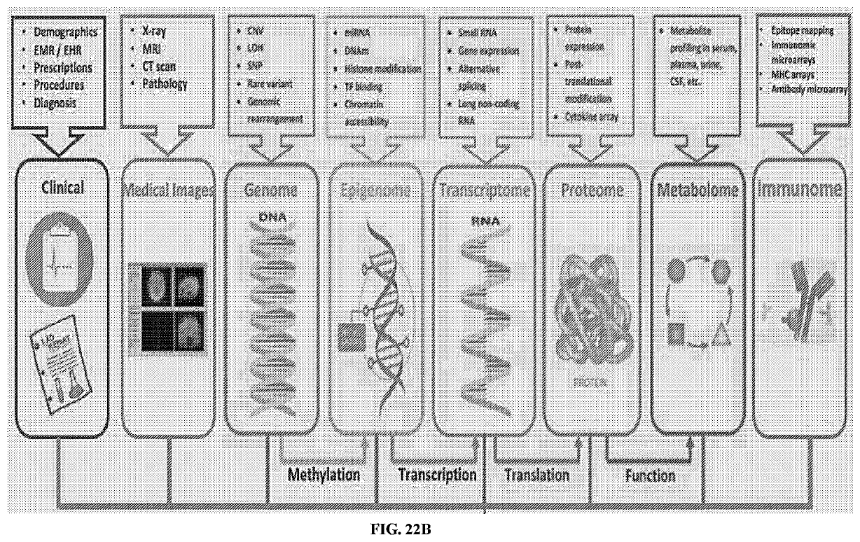

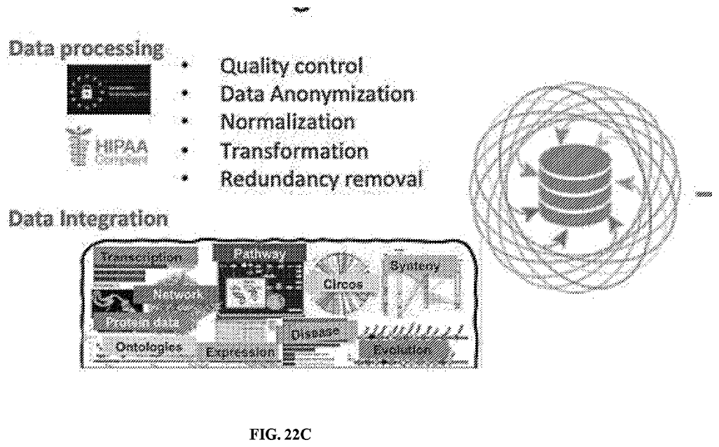

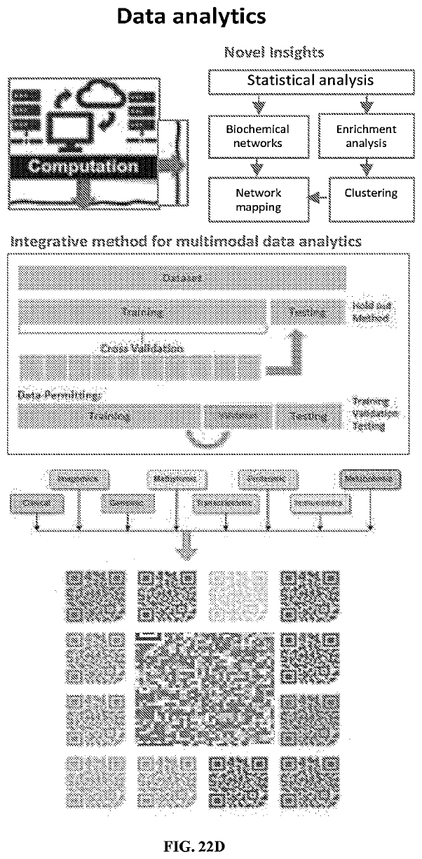

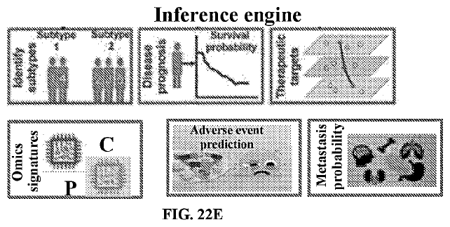

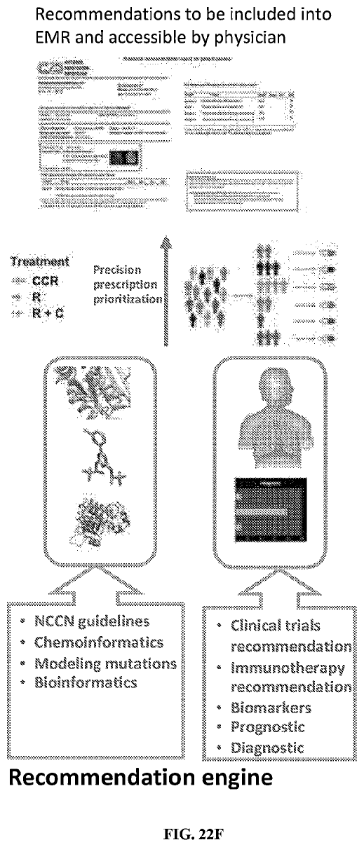

FIGS. 22A, 22B, 22C, 22D, 22E and 22F depict an example overview of the system for providing a recommendation of a prescription to a doctor for a patient. The system 2200 comprises a data acquisition module 2201 (as depicted in FIG. 22B), a data management module 2202 (as depicted in FIG. 22C), a data analytics module 2203 (as depicted in FIG. 22D), an Inference engine 2204 (as depicted in FIG. 22E), and a recommendation engine 2205 (as depicted in FIG. 22F).

The data acquisition module 2201 can receive a query from a user; in the depicted example, the user is a physician who requires a recommendation of a prescription for a user. The data acquisition module 2201 can acquire data, such as, but not limited to, clinical data, medical media, genome data, epigenome data, proteome data, metabolome data, and immunome data for the patient and similar cohorts. The acquired data can be then provided to the data management module 2202.

The data management module 2202 can perform one or more operations on the data, such as checking the data for quality, anonymizing the data, normalizing the data, transforming the data, removing redundant data, and so on. The data management module 2202 can integrate the data with the semantic ontology framework. This data can be provided to the data analytics module 2203 and the inference engine 2204. The data analytics module 2203 can perform multimodal analysis of the data, as depicted in FIG. 22D. The inference engine 2204 can receive inputs from the data management module 2202 and the data analytics module 2203, which can draw one or more inferences from the inputs. Examples of the inferences can be, metastasis probability, prediction of adverse events, providing therapeutic targets, disease prognosis (survival probability), identifying subtypes of the cohorts, and omics signatures. These inferences can be provided to the recommendation engine 2205.

The recommendation engine 2205 can use data such as, NCCN guidelines, chemoinformatics, modelling mutations, bioinformatics, clinical trial recommendation, immunotherapy recommendations, biomarkers, prognostic, diagnostic, and so on, for determining one or more recommendations. The recommendations can be in terms of, for example, precision medicine, prescription, prioritization, and so on. In this example, the recommendation is in the form of a prescription. These recommendations from the recommendation engine 2205 and inferences from the inference engine 2204 can be provided to the user.

Embodiments herein disclose a precision medicine ontology, which has been custom built with an aim to link transactional data and non-transactional data related to patients for precision decision support. Embodiments herein provide a unique way to see linked patient records. Embodiments herein can be used to design and implement precision medicine strategies for patient cohorts who do not respond to conventional treatments. Embodiments herein can serve as an accurate and intuitive recommender system for complex queries and real-time predictive analytics of static and real-time streaming data. Embodiments herein can identify the right patients for clinical trial enrolment and recruitment. Embodiments herein can simulate clinical trial study protocols prior to execution to optimize the time and cost involved in clinical R&D. Embodiments herein can serve as an instrument to create custom Ontology frameworks, Common Data Models, Ontology-based data catalog(s), and so on. Embodiments herein may also be used to grow and maintain the enterprise ontology framework. Embodiments herein can serve as a hand-held IOT device/scanner with a camera to capture images of diseased tissue/organs for real-time AI/ML based analytics. Embodiments herein can serve as a predictive platform for early onset of disease, temporal disease progression pathways, adverse events, optimal treatment regimens, clinical outcomes, clinical study end points for safety and efficacy evaluations, real-world observational study design and optimization. Embodiments herein can serve as predictive platform to aid healthcare reimbursement agencies to analyze patient claims datasets for insurance procedures and to analyze and approve prior authorization recommendations from healthcare providers (HCPs) for step-edit therapies or second-in-line or specialty therapies. Embodiments herein can help in drug R&D, in the discovery of new and novel therapeutic candidates. Embodiments herein can help in drug repositioning of safe and marketed pharmaceutical products.

The embodiments disclosed herein can be implemented through at least one software program running on at least one hardware device and performing network management functions to control the network elements. The network elements include blocks, which can be at least one of a hardware device, or a combination of hardware device and software module.

The embodiment disclosed herein specifies an enterprise graph-based, semantic ontology-based recommender framework. Therefore, it is understood that the scope of the protection is extended to such a program and in addition to a computer readable means having a message therein, such computer readable storage means contain program code means for implementation of one or more steps of the method, when the program runs on a server or mobile device or any suitable programmable device. The method is implemented in at least one embodiment through or together with a software program written in e.g., Very high speed integrated circuit Hardware Description Language (VHDL) another programming language, or implemented by one or more VHDL or several software modules being executed on at least one hardware device. The hardware device can be any kind of device which can be programmed including e.g., any kind of computer like a server or a personal computer, or the like, or any combination thereof, e.g., one processor and two FPGAs. The device may also include means which could be e.g., hardware means like e.g., an ASIC, or a combination of hardware and software means, e.g., an ASIC and an FPGA, or at least one microprocessor and at least one memory with software modules located therein. Thus, the means are at least one hardware means and/or at least one software means. The method embodiments described herein could be implemented in pure hardware or partly in hardware and partly in software. The device may also include only software means. Alternatively, the invention may be implemented on different hardware devices, e.g., using a plurality of CPUs.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments herein can be practiced with modification within the spirit and scope of the claims as described herein.

We claim:

1. A multi-modal, multi-omic method for providing clinical precision decision support for a patient, wherein the method comprises providing at least one recommendation to a user using an Artificial Intelligence (AI) model, and a semantic ontology recommender framework.

2. The method, as claimed in claim 1, wherein the semantic ontology recommender framework is a graph based semantic ontology recommender framework.

3. The method, as claimed in claim 1, wherein the method further comprises viewing the recommendation in a workspace.

4. The method, as claimed in claim 1, wherein the method further comprises viewing linked patient data and clinical journey of the patient in at least one of a text and a graphical format.

5. The method, as claimed in claim 1, wherein the method further comprises viewing, managing and analyzing cohort data using at least one of a text and a graphical format.

6. A multi-modal, multi-omic clinical precision decision support system for a patient, wherein the system provides at least one recommendation to a user using an Artificial Intelligence (AI) model, and a semantic ontology recommender framework.

7. The system, as claimed in claim 6, wherein the semantic ontology recommender framework is a graph based semantic ontology recommender framework.

8. The system, as claimed in claim 6, wherein the system enables users to view the recommendation in a workspace.

9. The system, as claimed in claim 6, wherein the system enables users to view linked patient data and clinical journey of the patient in at least one of a text and a graphical format.

10. The system, as claimed in claim 6, wherein the system enables users to view, manage and analyze cohort data using at least one of a text and a graphical format.
